(19) United States
(12) Patent Application Publication
(10) Pub. No.: US 2023/0014236 A1
(43) Pub. Date: Jan. 19, 2023

(54) Adult Hygiene Garment with Anti-Odor and Anti-Microbial Zones

(71) Applicant: Freida E. Abakah, Yukon, OK (US)

(72) Inventor: Freida E. Abakah, Yukon, OK (US)

(21) Appl. No.: 17/375,825

(22) Filed: Jul. 14, 2021

(51) Int. Cl. A61F 13/84; A61F 13/49; A61F 13/15; A61F 13/535

(52) U.S. Cl. CPC A61F 13/8405; A61F 13/49; A61F 13/15699; A61F 13/535; A61F 2013/8414; A61F 2013/847

(57) ABSTRACT

An adult hygiene garment has multiple flexible layers shaped to form a front panel and a rear panel joined by a perineum panel, in which the perineum panel is bounded on a left edge and on a right edge by two curved leg edges, and in which at least one of the layers comprises an anti-microbial anti-odor insert below an inner liner layer.

### FIELD OF THE INVENTION

This invention relates to hygienic garments for comfort, convenience and health for patients who are incontinent.

### BACKGROUND OF THE INVENTION

Adults who suffer from incontinence often suffer from maladies associated with urine and feces which are retained near the skin of an adult hygiene garment such as a disposable diaper. It is often not feasible to change the garment as soon as the wearer soils it due to immobility of the wearer, delays in getting health care assistant attention, or inconvenience of the situation or location. During the time between soiling and changing the garment, unpleasant odors may develop as may detrimental microbes such as bacteria, yeast and fungi. The latter may lead to irritation of the wearer's skin, and potentially to infections within the wearer.

### SUMMARY OF THE EXEMPLARY EMBODIMENTS OF THE INVENTION

An adult hygiene garment is disclosed which has multiple flexible layers shaped to form a front panel and a rear panel joined by a perineum panel, in which the perineum panel is bounded on a left edge and on a right edge by two curved leg edges, and in which at least one of the layers comprises an anti-microbial anti-odor insert below an inner liner layer.

### BRIEF DESCRIPTION OF THE DRAWINGS

The figures presented herein, when considered in light of this description, form a complete disclosure of one or more embodiments of the invention, wherein like reference numbers in the figures represent similar or same elements or steps.

FIG. 1 depicts a first embodiment according to the present invention in a flattened state.

FIG. 2 shows an example cross section of materials taken through the thickness along the midline of the example embodiment of FIG. 1.

### DETAILED DESCRIPTION

Soiled adult hygiene garments are a known source of odor, irritation and infection, and yet, available products remain unimproved to inhibit and prevent these undesirable consequences of wearing such products. The present inventor, through her work as a nurse and healthcare services professional, has developed an improved adult hygiene garment which addresses these unmet needs in the art, and is particularly well suited for production as a disposable garment with low production costs.

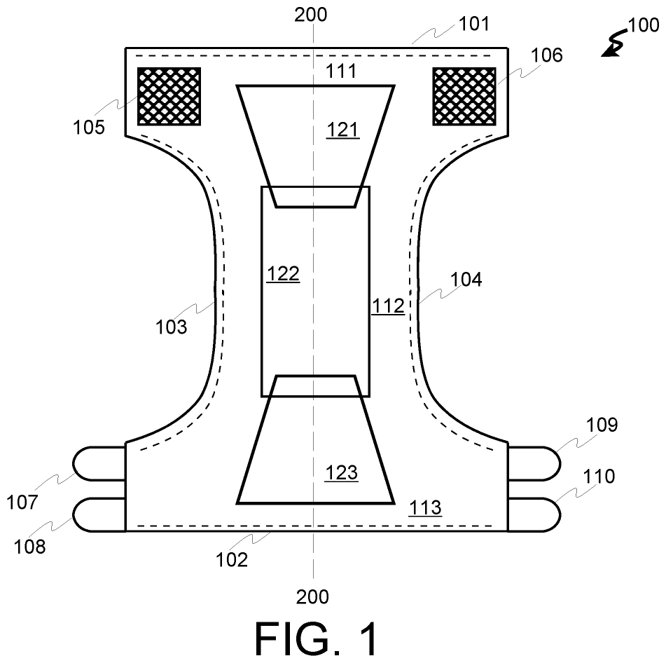

Turning to FIG. 1, a first embodiment 100 according to the present invention is shown in a flattened state. For convention, we will refer to a front edge 101 and a rear edge 102 which generally are arranged in parallel and opposition to each other. Between the front and rear edges are a left leg edge 103 and a right leg edge 104, between which is longitudinally formed a crotch or perineum panel 112 which extends laterally between a front panel 111 and a rear panel 113. Stitching or expandable gathering is preferably provided along the front and back edges and along the leg edges as shown by the dashed lines to improve fit and conformance to the wearer when applied. One or more closure tabs 107, 108, 109, and 110 are provided to align with one or more mating closure panels 105, 106, such as a hook-and-look fastener pairs, to connect the sides of the front and rear panels to each other when worn by a wearer.

These components of the example garment resemble an ordinary adult hygiene garment, albeit in at least one embodiment, the dimensions are increased to accommodate wearing by persons having a large waist size (example dimensions will be discussed in the following paragraphs).

According to one or more improvements in this embodiment, one or more hygienic inserts are added to the groin area 121, perineum area 122, or buttocks area 123 in the front panel 111, perineum panel 112, or rear panel 113, respectively. In a preferred embodiment, the groin hygienic insert 121 and the buttocks hygienic insert 123 are provided with a trapezoid shape, as illustrated, wherein the groin hygienic insert 121 is especially applicable for male wearers and the other inserts 122 and 123 are applicable to both male and female wearer. Although the insert shapes are shown with sharp corners, other embodiments may include rounded corners, ovals and oblong shapes.

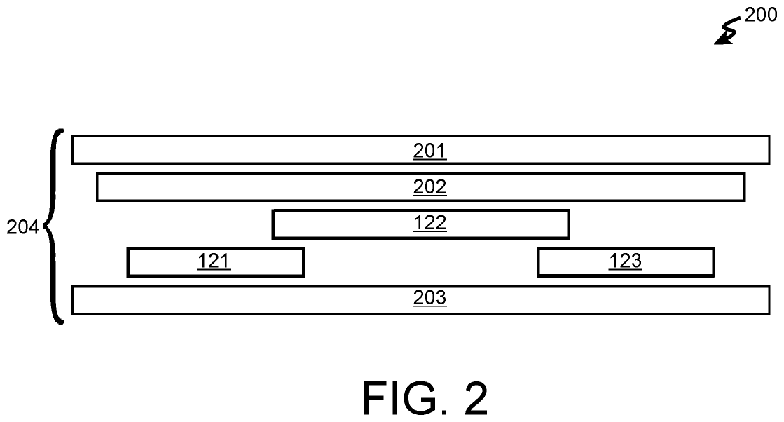

Referring now to FIG. 2, an example cross section of the materials taken through the thickness 204 along the midline 200 of FIG. 1 is shown. Please note that the thickness of the materials is not shown to scale in this depiction. The inside liner 201, which contacts the skin of the wearer, is of a conventional material such as a moisture barrier which allows moisture to travel from the wearer's skin to the inner layers 202, 121, 122 and 123, but which tends to prevent reverse movement of moisture from the inner layers back towards the wearer's skin. A moisture absorbing layer 202 is disposed beneath the inner liner 201, such as but not limited to Super Absorbent Polymers (SAP) gel, cotton, hemp, bamboo, microfiber, polylactice acid (PLA) fibers, or polyurethane (PU) fibers. An outer shell 203 provides the visible exterior to the garment while worn, and may be made of any suitable material ranging from natural fiber cloth to synthetic fiber cloth to non-woven sheet material such as polyvinyl chloride (PVC), and others.

The improvement includes one, two or three inserts 121, 122, and 123 made of material which is anti-odor and anti-microbial, and which may overlap each other in some embodiments, or which may be combined into just one or two inserts in other embodiments. While the embodiment example of FIG. 1 shows three distinct separate inserts for the convenience of defining zones, in practice and in some embodiments, these inserts may be combined into two or just one physical insert which occupy one, two or three of the defined zones. The material used to fabricate the insert(s) may be of a variety of known anti-odor and/or anti-microbial materials. One such material is sheet material and woven materials infused with nano-silver, far-infrared ray or anion elements (chips), or a combination thereof. Methods and chemical formulae for producing such materials, including their verified biological benefits, such as those described in “Antimicrobial silver: An unprecedented anion effect” by Swathy et al. (Scientific Reports, Nov. 24, 3014) and “Multifunctional Hydroxyapatite/Silver Nanoparticles/Cotton Gauze for Antimicrobial and Biomedical Applications” by Said et al. (Nanomaterials, Feb. 8, 2021) may be employed to yield suitable insert material.

Other such materials suitable for fabricating the inserts are various plastic fibers and films available with SILVERSHIELD™ or zinc-based ZPTECH (zinc), both available from Microban USA of Huntersville, N.C., such as treated Acrylonitrile Butadiene Styrene (ABS), Polypropylene (PP), Polystyrene (PS), Polyethylene (PE/LDPE), Polyvinyl chloride (PVC), and Polycarbonate (PC). Additional odor control can be obtained by optionally including in the material of the inserts activated carbon.

Improved adult dimensions according to at least one embodiment include front and rear edges which are between 12 and 24 inches each in a stretched (ungathered) state allowing for a fit around a waist of 24 to 48 inches, with 8 inch tall front and rear panels, and a 12 inch perineum portion extending between the front and rear panels. The leg edges are preferably a smooth curve which indents about 3 inches (ungathered) from the right and left edges of the front and rear panels.

The terminology used herein is for the purpose of describing particular exemplary embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof, unless specifically stated otherwise.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated. The foregoing example embodiments do not define the extent or scope of the present invention, but instead are provided as illustrations of how to make and use at least one embodiment of the invention.

I/We claim:

1. An adult hygiene garment comprising multiple flexible layers shaped to form a front panel and a rear panel joined by a perineum panel, wherein the perineum panel is bounded on a left edge and on a right edge by two curved leg edges, and in which at least one of the multiple flexible layers comprises an anti-microbial anti-odor insert below an inner liner layer.

2. The adult hygiene garment as set forth in claim 1 wherein the anti-microbial anti-odor insert comprises an elongated insert extending a length of the perineum panel.

3. The adult hygiene garment as set forth in claim 1 wherein the anti-microbial anti-odor insert comprises an trapezoid-shaped insert placed in a groin area of the front panel.

4. The adult hygiene garment as set forth in claim 1 wherein the anti-microbial anti-odor insert comprises an trapezoid-shaped insert placed in a buttock area of the rear panel.

5. The adult hygiene garment as set forth in claim 1 wherein the anti-microbial anti-odor insert comprises a material infused with nano-silver.

6. The adult hygiene garment as set forth in claim 1 wherein the anti-microbial anti-odor insert comprises a material embedded with far-infrared ray chip.

7. The adult hygiene garment as set forth in claim 1 wherein the anti-microbial anti-odor insert comprises a material embedded with negative ion (anion) chip.

8. The adult hygiene garment as set forth in claim 7 wherein the negative ion chip comprises a zinc material.

9. The adult hygiene garment as set forth in claim 1 wherein the anti-microbial anti-odor insert comprises a material embedded with activated carbon.

10. The adult hygiene garment as set forth in claim 1 wherein the anti-microbial anti-odor insert comprises one or more treated materials selected from the group consisting of Acrylonitrile Butadiene Styrene (ABS), Polypropylene (PP), Polystyrene (PS), Polyethylene (PE/LDPE), Polyvinyl chloride (PVC), and Polycarbonate (PC).

11. A method of manufacture of an adult hygiene garment comprising producing multiple flexible layers shaped to form a front panel and a rear panel joined by a perineum panel, wherein the perineum panel is bounded on a left edge and on a right edge by two curved leg edges, and in which at least one of the multiple flexible layers comprises an anti-microbial anti-odor insert below an inner liner layer.

12. The method of manufacture as set forth in claim 11 wherein the anti-microbial anti-odor insert comprises an elongated insert extending a length of the perineum panel.

13. The method of manufacture as set forth in claim 11 wherein the anti-microbial anti-odor insert comprises an trapezoid-shaped insert placed in a groin area of the front panel.

14. The method of manufacture as set forth in claim 11 wherein the anti-microbial anti-odor insert comprises an trapezoid-shaped insert placed in a buttock area of the rear panel.

15. The method of manufacture as set forth in claim 11 wherein the anti-microbial anti-odor insert comprises a material infused with nano-silver.

16. The method of manufacture as set forth in claim 11 wherein the anti-microbial anti-odor insert comprises a material embedded with far-infrared ray chip.

17. The method of manufacture as set forth in claim 11 wherein the anti-microbial anti-odor insert comprises a material embedded with negative ion (anion) chip.

18. The method of manufacture as set forth in claim 17 wherein the negative ion chip comprises a zinc material.

19. The method of manufacture as set forth in claim 11 wherein the anti-microbial anti-odor insert comprises a material embedded with activated carbon.

20. The method of manufacture as set forth in claim 11 wherein the anti-microbial anti-odor insert comprises one or more treated materials selected from the group consisting of Acrylonitrile Butadiene Styrene (ABS), Polypropylene (PP), Polystyrene (PS), Polyethylene (PE/LDPE), Polyvinyl chloride (PVC), and Polycarbonate (PC).
